(19) United States
(12) Patent Application Publication
(10) Pub. No.: US 2016/0158305 A1
(43) Pub. Date: Jun. 9, 2016

(54) ADDITIVES AND SUPPLEMENTS

(71) Applicant: Chip E. THOMSON, Austin, TX (US)

(72) Inventor: Chip E. THOMSON, Austin, TX (US)

(21) Appl. No.: 14/858,212

(22) Filed: Sep. 18, 2015

Related U.S. Application Data
(60) Provisional application No. 62/052,748, filed on Sep. 19, 2014.

(51) Int. Cl. A61K 36/82; A61K 31/385; A61K 31/015; A61K 31/122; C12G 3/04; A61K 31/714; A61K 31/519; A61K 9/16; A61K 36/28; A61K 31/401; A61K 31/198; A61K 31/14

(52) U.S. Cl. CPC A61K 36/82; A61K 31/198; A61K 31/385; A61K 31/015; A61K 31/122; A61K 31/14; A61K 31/714; A61K 31/519; A61K 9/16; A61K 36/28; A61K 31/401; A23L 1/3051; A23L 1/302; A23L 1/3002; C12G 3/04; A23V 2002/00

(57) ABSTRACT

Disclosed are ingestible additives and ingestible supplements that are capable of promoting liver function, promoting liver health, or reducing alcoholic hepatopathy in a subject. The ingestible additive or supplement can include at least one essential amino acid, at least non-essential amino acid, at least one antioxidant, at least one lipotropic compound, at least one water-soluble vitamin, or at least one plant or extract thereof.

### CROSS-REFERENCE TO RELATED APPLICATIONS

This application claims the benefit of U.S. Provisional Application No. 62/052,748 filed on Sep. 19, 2014. The contents of the referenced application are incorporated into the present application by reference.

### BACKGROUND OF THE INVENTION

A. Field of the Invention

The present invention relates generally to additives and supplements that can be used with alcoholic beverages. The additives and supplements can help ameliorate the negative effects that alcohol can have on a person's liver function and health.

B. Description of the Related Art

In today's society, it is well known that alcohol can adversely affect the function and health of a person's liver. Short-term and long-term liver damage caused by alcohol is well characterized. For instance, alcohol-induced liver diseases and conditions, or alcoholic hepatopathy, can result in fatty liver, hepatitis, hepatic fibrosis, hepatic cirrhosis, carcinoma of liver, etc.

While some attempts have been made to produce ingredients that can help protect a person's liver function and health from the deleterious effects of alcohol, such attempts have been inadequate. The level of protection offered by such ingredients has been limited in their efficacy as well as their solubility within an alcoholic beverage.

### SUMMARY OF THE INVENTION

Solutions to the current problems associated with the use of additives and supplements in alcoholic beverages has been discovered. The solution is premised on the use of various classes of ingredients that work together to promote a healthy liver while also protecting the liver from the potential damaging effects caused by alcohol. The ingredients can include essential amino acids, non-essential amino acids, antioxidants, lipotropic compounds, water-soluble vitamins, or plants or extracts thereof, or combinations thereof. In particular, it has been discovered that various combinations of ingredients within these classes can be used to promote liver function and liver health. By way of example, these combinations can be used to reduce, delay, or prevent the onset of liver diseases. Without wishing to be bound by theory, it is believed that the combination of ingredients, along with the ingredients' respective water-solubility characteristics, provides the liver with a sufficient amount of nutrients to counteract the negative effects that alcohol has on liver function and health.

In one non-limiting aspect of the present invention there is disclosed an additive or supplement capable of reducing alcoholic hepatopathy in a subject, the additive or supplement comprising at least one essential amino acid, at least non-essential amino acid, at least one antioxidant, at least one lipotropic compound, at least one water-soluble vitamin, or at least one plant or extract thereof. In particular embodiments, the additive or supplement can include an ingredient from at least 2, 3, 4, 5, or all 6 classes of the following ingredients: an essential amino acid; a non-essential amino acid; an antioxidant; a lipotropic compound; a water-soluble vitamin; and a plant or extract thereof. For example, the additive or supplement can include an essential amino acid and a non-essential amino acid. The additive or supplement can include an essential amino acid and an antioxidant. The additive or supplement can include an essential amino acid and a lipotropic compound. The additive or supplement can include an essential amino acid and a water-soluble vitamin. The additive or supplement can include an essential amino acid and a plant or extract thereof. The additive or supplement can include a non-essential amino acid and an antioxidant. The additive or supplement can include a non-essential amino acid and a lipotropic compound. The additive or supplement can include a non-essential amino acid and a water-soluble vitamin. The additive or supplement can include a non-essential amino acid and a plant or extract thereof. The additive or supplement can include an antioxidant and a lipotropic compound. The additive or supplement can include an antioxidant and a water-soluble vitamin. The additive or supplement can include an antioxidant and a plant or extract thereof. The additive or supplement can include a lipotropic compound and a water-soluble vitamin. The additive or supplement can include a lipotropic compound and a plant or extract thereof. The additive or supplement can include a water-soluble vitamin and a plant or extract thereof. The additive or supplement can include an essential amino acid, a non-essential amino acid, and an antioxidant. The additive or supplement can include an essential amino acid, a non-essential amino acid, and a lipotropic compound. The additive or supplement can include an essential amino acid, a non-essential amino acid, and a water-soluble vitamin. The additive or supplement can include an essential amino acid, a non-essential amino acid, and a plant or extract thereof. The additive or supplement can include a non-essential amino acid, an antioxidant, and a lipotropic compound. The additive or supplement can include a non-essential amino acid, an antioxidant, and a water-soluble vitamin. The additive or supplement can include a non-essential amino acid, an antioxidant, and a plant or extract thereof. The additive or supplement can include an antioxidant, a lipotropic compound, and a water-soluble vitamin. The additive or supplement can include an antioxidant, a lipotropic compound, and a plant or extract thereof. The additive or supplement can include a lipotropic compound, a water-soluble vitamin, and a plant or extract thereof. The additive or supplement can include an essential amino acid, a non-essential amino acid, an antioxidant, and a lipotropic compound. The additive or supplement can include an essential amino acid, a non-essential amino acid, an antioxidant, and a water-soluble vitamin. The additive or supplement can include an essential amino acid, a non-essential amino acid, an antioxidant, and a plant or extract thereof. The additive or supplement can include an essential amino acid, a non-essential amino acid, an antioxidant, a lipotropic compound, and a water-soluble vitamin. The additive or supplement can include an essential amino acid, a non-essential amino acid, an antioxidant, a lipotropic compound, and a plant or extract thereof. The additive or supplement can include an essential amino acid, a non-essential amino acid, an antioxidant, a lipotropic compound, a water-soluble vitamin, and a plant or extract thereof. It is noted that salt forms of each aforementioned class of ingredients are contemplated. In even more particular embodiments, the additive or supplement includes an ingredient from each of these classes of ingredients (e.g., an essential amino acid, a non-essential amino acid, an antioxidant, a lipotropic compound, a water-soluble vitamin, and a plant or extract thereof). In certain embodiments, the additive or supplement is capable of being partially or fully solubilized in the alcoholic beverage. Further, the additive or supplement can be odorless and tasteless once solubilized in the beverage such that it does not affect the taste or smell of the beverage. The additive or supplement can include at least two, three, four, five, six, seven, or eight essential amino acids selected from histidine, isoleucine, leucine, lysine, methionine, phenylalanine, threonine, tryptophan, or valine.

In one particular embodiment, a supplement is contemplated. A supplement is a product, ingredient, composition, etc., that can be taken prior to, simultaneously with, or after consumption of an alcoholic beverage, or combinations thereof. By way of example, a supplement can be in the form of a pill, tablet, gel cap, a liquid, a spray, a powder, or other ingestible product etc. In particularly preferred embodiments, the supplement is a gel cap. In one instance, the supplement can be taken prior to and during alcohol consumption. In another instance, the supplement can be taken prior to and after alcohol consumption. In a further instance, the supplement can be taken prior to and after alcohol consumption. In still another instance, the supplement can be taken during and after alcohol consumption. In yet another instance, the supplement can be taken prior to, during, and after alcohol consumption. In one particular embodiment, the supplement is taken daily irrespective of whether the user has an alcoholic beverage. In another particular embodiment, when a user anticipates having an alcoholic beverage for a given day, the user can take the supplement in the morning followed up with another supplement up to 4 hours (preferably 2 to 4 hours) prior to having an alcoholic beverage, followed with yet another supplement within 4 hours (preferably 2 to 4 hours) after ingesting/having an alcoholic beverage. In certain instances, the user takes a maximum of four supplements in one day. In other instances, at least one supplement can be taken within 24 hours, within 20 hours, within 15 hours, within 10 hours, within 5 hours, 4 hours, within 3 hours, within 2 hours, within 1 hour, within 30 minutes, within 15 minutes, within 10 minutes, within 5 minutes or less prior to consuming an alcoholic beverage, preferably within 4 hours of prior to consuming an alcoholic beverage, and most preferably within 2 to 4 hours prior to consuming an alcoholic beverage. Further, at least one supplement can be taken within 24 hours, within 20 hours, within 15 hours, within 10 hours, within 5 hours, 4 hours, within 3 hours, within 2 hours, within 1 hour, within 30 minutes, within 15 minutes, within 10 minutes, within 5 minutes or less after consuming an alcoholic beverage, preferably within 4 hours after consuming an alcoholic beverage, and most preferably within 2 to 4 hours after consuming an alcoholic beverage. In a particular instance, the additive includes alpha lipoic acid, beta carotene, choline, curcumin, cysteine, glutamine, glycine or taurine or both, folic acid, Camellia sinensis extract, inositol, methylsulfonylmethane (MSM), Silibum marianum extract, s-adenosyl methionine (SAM-e), vitamin C, vitamin E (e.g., tocopherols and tocotrienols, preferably α-tocopherol, or salt forms thereof (e.g., tocopherol acetate)), and zinc gluconate. In a particular embodiment, the supplement includes 5 mg to 15 mg of alpha lipoic acid, 3 mg to 7 mg of beta carotene, 5 mg to 15 mg of choline, 15 mg to 25 mg of curcumin, 25 mg to 35 mg of cysteine, 75 mg to 125 mg of glutamine, 1 mg to 125 mg of glycine or taurine or 75 mg to 125 mg of a mixture having glycine and taurine, 225 mcg to 275 mcg of folic acid, 175 mg to 225 mg of Camellia sinensis extract, 5 mg to 15 mg of inositol, 75 mg to 125 mg of methylsulfonylmethane (MSM), 225 mg to 275 mg of Silibum marianum extract, 75 mg to 125 mg of s-adenosyl methionine (SAM-e), 50 mg to 70 mg of vitamin C, 25 IU to 35 IU of vitamin E (e.g., tocopherols and tocotrienols, preferably α-tocopherol, or salt forms thereof (e.g., tocopherol acetate)), and 3 mg to 7 mg of zinc gluconate. In an even more particular instance, the supplement includes 10 mg of alpha lipoic acid, 5 mg beta carotene, 10 mg of choline, 20 mg of curcumin, 30 mg of cysteine, 100 mg of glutamine, 100 mg of a mixture comprising glycine and taurine, 250 mcg of folic acid, 200 mg of Camellia sinensis extract, 10 mg of inositol, 100 mg of methylsulfonylmethane (MSM), 250 mg of Silibum marianum extract, 100 mg of s-adenosyl methionine (SAM-e), 60 mg of vitamin C, 30 IU of vitamin E (e.g., tocopherols and tocotrienols, preferably α-tocopherol, or salt forms thereof (e.g., tocopherol acetate)), and 5 mg of zinc gluconate. The supplement can be substantially similar to the formulation provided at Table 2. In a particular embodiment, the supplement can be comprised in a capsule. The capsule can be a gelatin capsule (Gel Cap). The supplement can be in powdered form in the capsule. The supplement can be in liquid form in the capsule.

In another particular instance, an additive is contemplated. Similar to a supplement, an additive can be a product, ingredient, composition, etc., that is separate from an alcoholic beverage or other ingestible product. However, the additive is added or mixed into an alcoholic beverage or ingestible product. For example, this adding/mixing can be done by simply adding the additive to an alcoholic beverage prior to or during consumption of said beverage. It can be added by the person consuming the beverage, by a waiter, by a bartender, by the manufacturer of an alcoholic beverage, etc. Therefore, the additive is consumed simultaneously with the alcoholic beverage. In one particular embodiment, the additive includes histidine, isoleucine, leucine, lysine, methionine, tryptophan, and valine. The additive can include 250 mg to 350 mg of histidine, 400 mg to 600 mg of isoleucine, 400 mg to 600 mg of leucine, 400 mg to 600 mg of lysine, 40 mg to 60 mg of methionine, 5 mg to 15 mg of tryptophan, and 150 mg to 250 mg of valine. The amounts of each ingredient can be modified as desired to include less or more of the stated amounts. The additive can also include a combination of essential amino acids that consists of histidine, isoleucine, leucine, lysine, methionine, tryptophan, and valine. In some instances, the additive includes at least two, three, four, five, six, seven, eight, nine, ten, or eleven non-essential amino acids selected from alanine, arginine, aspartic acid, cysteine, glutamic acid, glutamine, glycine, proline, serine, tyrosine, asparagine, or selenocysteine. The additive can include alanine, aspartic acid, cysteine, glutamine, glycine, proline, serine, and tyrosine. In one aspect, the additive can include 250 mg to 350 mg of alanine, 250 mg to 350 mg of aspartic acid, 20 mg to 40 mg of cysteine, 450 mg to 550 mg of glutamine, 50 mg to 150 mg of glycine, 150 mg to 250 mg of proline, 250 mg to 350 mg of serine, and 150 mg to 250 mg of tyrosine. The amounts of each ingredient can be modified as desired to include less or more of the stated amounts. In one instance, additive comprises a combination of non-essential amino acids that consists of alanine, aspartic acid, cysteine, glutamine, glycine, proline, serine, and tyrosine. In a particular instance, the additive can include an antioxidant. A non-limiting example of an antioxidant includes alpha lipoic acid. Other antioxidants can be used in the context of the present invention. The additive can include 20 to 40 mg of alpha lipoic acid or additional amounts below and above the stated range as desired. The additive can include at least one lipotropic compound. Non-limiting examples of lipotropic compounds include choline or inositol or a combination thereof. The additive can include 5 mg to 15 mg of choline and 15 mg to 25 mg of inositol. The amounts of each ingredient can be modified as desired to include less or more of the stated amounts. In some instances, the additive can include a water-soluble vitamin. Non-limiting examples include vitamin C or vitamin B. Examples of vitamin B include vitamins B₁, B₂, B₃, B₅, B₆, B₇, B₉, and B₁₂. In some particular aspects, the combination of vitamin C, vitamin B₉ (e.g., folic acid or folate) and vitamin B₁₂ are included in the additive. The amounts within the additive can include 50 mg to 70 mg of vitamin C, 400 mg to 600 mg of vitamin B₁₂, and 300 mg to 500 mg of vitamin B₉. The additive can include at least one plant or extract thereof. Non-limiting examples of plants and extracts thereof include Silybum marianum or Camellia sinensis or a combination thereof. The plant can be any portion thereof (e.g., leaf, stem, root, bud, flower, fruit, etc.), the whole plant, or an extract of said portion or whole plant. In particular instances, the additive includes Silybum marianum fruit extract and Camellia sinenis leaf extract. The extractant used to obtain the extract can be water, alcohol, a combination of water and alcohol (i.e., hydro alcoholic), a glycol, or a hydro glycol. In particular embodiments, the extractant used is an aqueous or alcoholic extract or a hydro alcoholic extract. The amounts of these plants or extracts within the additive can be 250 mg to 350 mg of Silybum marianum or an extract thereof and 100 mg to 200 mg of Camellia sinensis or an extract thereof. The amounts of each ingredient can be modified as desired to include less or more of the stated amounts. Additional plants and extracts thereof that can be used include Vitis vinifera (e.g., Vitis vinifera seed or an extract thereof). The additive can also include curcumin. Other ingredients that can be used in the extract include methylsulfonylmethane and zinc acetate. The amounts within the additive can be 400 mg to 600 mg of methylsulfonylmethane and 10 mg to 20 mg of zinc acetate. The amounts of each ingredient can be modified as desired to include less or more of the stated amounts. In certain instances, the additives of the present invention can be in tablet form, liquid form, or powdered form. Other forms include pastes, granules, syrups, lozenges, capsules, or the like. In more particular aspects, the additive can be in a powdered form. The additive can be fully solubilized in the alcoholic beverage. Additional ingredients, excipients, and preservatives can also be included in the additive. Further, and in one instance, the additive can include the ingredients listed in Table 1. It can also include the ingredients listed in Table 1 in the amounts also listed in Table 1. In addition to the ingredients discussed in the summary of the invention section of the specification, the ingredients identified in the detailed description can also be included in the additives of the present invention.

In yet another aspect of the present invention, there is disclosed a composition comprising any one of the additives or supplements of the present invention. The composition can be an edible composition. The edible composition can be a food, a beverage, or a medicinal formulation. In more particular aspects, the composition is an alcoholic beverage when an additive is used. The composition, such as an alcoholic beverage, can have a reduced hepatopathic effect on a subject's liver when compared with the same composition (e.g., alcoholic beverage) that does not include the additive. In instances where the composition is an alcoholic beverage, the alcoholic beverage can include 0.5% to 50% alcohol by volume (ABV). In some instances, it can include more than 50%, 60%, 70%, 80%, 90%, or more ABV. In one instance, the alcoholic beverage can be a beer or wine. The alcoholic content of the beer or wine can be 0.5% to 20% alcohol by volume (ABV). In some instances, the alcoholic beverage can include a liquor. Non-limiting examples of liquor include vodka, gin, tequila, rum, whisky (e.g., scotch or bourbon or both), brandy, baijiu, soju, aguardiente, palinka, fernet, or slivovitz, or any combination thereof. In still other instances, the additive can be placed directed into the liquor or the liquor itself can be the alcoholic beverage. In addition to alcohol, the alcoholic beverage can include other ingredients (e.g., fruit, syrups, flavoring agents, additional non-alcoholic liquids (e.g., soda), etc.). The liquor can include at least 20% alcohol by volume (ABV) of the liquor. The additive can be partially or fully solubilized in the food, drink or beverage, or medicinal formulation. In some instances, 0.1 g to 10 g or 0.5 to 5 g of additive are comprised in the food, beverage, or medicinal composition. The amount added to the food, beverage, or medicinal composition can be up to the solubility limit of the additive within the particular food, beverage, or medicinal composition. The solubility limit can be ascertained by included the additive into the composition at room temperature (20 to 25° C.) under mixing conditions until the additive precipitates out of the composition. In particular instances, the ingredients in the additive are water-soluble or capable of dissolving in water.

Also disclosed in the context of the present invention is a method of reducing the hepatopathic effect of food, beverage, or medical composition on a subject's liver, the method comprising administering or ingesting any one of the supplements of the present invention prior to, during, and/or after, consuming the food, beverage, or medicinal composition. Still further, there is disclosed in the context of the present invention a method of reducing the hepatopathic effect of food, beverage, or medical composition on a subject's liver, the method comprising adding any one of the additives of the present invention to the food, beverage, or medicinal composition. In particular instances, the composition is an alcoholic beverage. However, many food products (e.g., sugary foods (e.g., ice cream, candy, etc.), salty foods (e.g., potato chips, french fries, canned foods, etc.), dairy products (e.g., milk, cheese, yogurt, etc.)) and medicinal formulations (e.g., oral liquid formulations having drugs such as acetaminophen, salicylates, acebutolol, indomethacin, etc.) have similar negative effects on liver function and health. Thus, the use of the additives or supplements of the present invention are not limited to alcoholic beverages. Rather, the additives or supplements can be used to food products and medicinal products that are also ingestible and that are known or are later discovered to cause hepatopathy.

Still further there is disclosed a method of reducing the hepatopathic effect of a food, beverage, or medicinal composition on a subject's liver, the method comprising ingesting a food, beverage, or medicinal composition comprising any one of the additives of the present invention or ingesting any one of the supplements of the present invention prior to, during, or after ingesting the food, beverage or medicinal composition. In particular instances, the composition is an alcoholic beverage.

Even further there is disclosed a method of making any one of the food, beverage, or medicinal compositions of the present invention, the method comprising mixing any one of the additives of the present invention with the food, beverage, or medicinal composition. In particular instances, the composition is an alcoholic beverage.

In another aspect of the present invention there is disclosed a kit comprising any one of the additives or supplements of the present invention. The kit can include a container and the additive or supplement placed within the container. The container can be a plastic container, a paper or cloth based container (e.g., approximately the size and shape of a standard sugar bag or salt package, etc.), a solid container that dissolves in an aqueous or alcoholic environment (e.g., a capsule that dissolves away in the presence of water or alcohol and releases the additive). Instructions can be included with the kit. For example, the instructions can state that the additive can be added to a food, beverage, or medicinal composition and mixed prior to ingesting the food, beverage or medicinal composition. The instructions can state that the supplement can be ingested prior to, during, or after consumption of a food, beverage, or medicinal composition. Again, the preferred composition is an alcoholic beverage.

Reference to “essential amino acid” includes the essential amino acid or its salt form. Reference to “non-essential amino acid” includes the non-essential amino acid or its salt form. Reference to “antioxidant” includes the antioxidant or its salt form. Reference to “lipotropic compound” includes the lipotropic compound and its salt form. Reference to “water-soluble vitamin” includes the water-soluble vitamin and its salt form. Reference to “plant” includes the plant (or any part of the plant) and an extract obtained from the plant (or any part of the plant). Reference to “oil-soluble vitamin” includes the oil-soluble vitamin and its salt form.

“Hepatopathy” refers to an abnormal or disease state of the liver (e.g., fatty liver, hepatitis, hepatic fibrosis, hepatic cirrhosis, carcinoma of liver, etc.). “Alcoholic hepatopathy” refers to an abnormal or disease state of the liver caused by ingestion of alcoholic beverages.

“Subject” refers to a mammal (e.g., human, primate, dog, cat, bovine, ovine, porcine, equine, mouse, rate, hamster, rabbit, or guinea pig). In particular embodiments, the subject is a human.

“Inhibiting” or “reducing” or any variation of these terms includes any measurable decrease or complete inhibition to achieve a desired result.

“Effective” or “treating” or “preventing” or any variation of these terms means adequate to accomplish a desired, expected, or intended result.

“Fully solubilized” refers to the additive being completely dissolved in the food, beverage, or medicinal composition such that there is no longer particulate matter visible (e.g., the composition can be described as optically clear with respect to the additive).

“Derivative,” “Analogue,” and “analog,” when referring to an ingredient of the present invention refers to a chemically modified form of the ingredient, wherein at least one substituent is not present in the parent ingredient. One such non-limiting example is vitamin C that has been covalently modified (e.g., ascorbyl phosphate).

“About” or “approximately” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The additives and compositions and methods of the present invention can “comprise,” “consist essentially of,” or “consist of” any of the ingredients or steps disclosed throughout the specification. With respect to the transitional phase “consisting essentially of,” in one non-limiting aspect, a basic and novel characteristic of the additives, supplements, compositions, and methods of the present invention is their ability to reduce the occurrence of hepatopathy.

Other objects, features, and advantages of the present invention will be apparent to one of skill in the art from the following detailed description and claims.

### DETAILED DESCRIPTION

The present invention provides an additive or supplement that can be used, for example, to prevent or reduce alcoholic hepatopathy. It is believed that the ingredients used in the additives or supplements of the present invention, as well as the solubility of the additive or supplement in aqueous and alcoholic environments, results in a product that can efficiently provide nutrients to the liver and improve the overall health and function of the liver. This can result in reducing the likelihood of developing liver diseases or conditions associated with consuming alcoholic beverages (e.g., fatty liver, hepatitis, hepatic fibrosis, hepatic cirrhosis, carcinoma of liver, etc.). Notably, the additives of the present invention can be used as a component in alcoholic beverages to produce alcoholic beverages having reduced hepatopathic effects. Supplements can be taken prior to, during, or after consumption of an alcoholic beverage, and act to promote liver function and liver health.

These and other non-limiting aspects of the present invention are discussed in the following subsections.

### A. Ingredients for the Additives and Supplements

The ingredients that can be included in the additives and supplements of the present invention include ingredients that can have a beneficial impact on the health or function of the liver as well as inactive excipients, binders, preservatives, etc.

Non limiting examples of ingredients that can be beneficial to liver function and health include essential amino acids, non-essential amino acids, antioxidants, lipotropic compounds, water-soluble vitamins, and plants or extracts thereof. Essential amino acids include amino acids that the human body cannot synthesize on its own. These include histidine, isoleucine, leucine, lysine, methionine, phenylalanine, threonine, tryptophan, and valine. Essential amino acids are commercially available from a wide range of sources (e.g., Sigma-Aldrich Co. LLC, St. Louis, Mo.). As noted above, “essential amino acids” refers to native amino acids and there corresponding salt forms. Derivatives and analogues of essential amino acids are also contemplated as being useful in the context of the present invention. In particular embodiments of the present invention, the selection of essential amino acids to include in the additives and supplements can be based on the effects that a given essential amino acid has on liver function or protection and the solubility of the essential amino acid in alcoholic and aqueous environments.

Non-essential amino acids include amino acids that can be synthesized by the body. Non-essential amino acids include alanine, arginine, aspartic acid, cysteine, glutamic acid, glutamine, glycine, proline, serine, tyrosine, asparagine, and selenocystein. Non-essential amino acids are commercially available from a wide range of sources (e.g., Sigma-Aldrich Co. LLC, St. Louis, Mo.). As noted above, “non-essential amino acids” refers to native amino acids and there corresponding salt forms. Derivatives and analogues of non-essential amino acids are also contemplated as being useful in the context of the present invention. In particular embodiments of the present invention, the selection of non-essential amino acids to include in the additives and supplements can be based on the effects that a given non-essential amino acid has on liver function or protection and the solubility of the non-essential amino acid in alcoholic and aqueous environments.

Antioxidants are substances that can inhibit the oxidation of other molecules and tissue (e.g., liver tissue). There are a wide range of antioxidants that are commercially available in the market and that can be used in the context of the present invention. A non-limiting example of an antioxidant includes alpha lipoic acid, which is commercially available from Sigma-Aldrich Co. LLC, St. Louis, Mo. Other non-limiting examples of antioxidants include vitamin C and its derivatives, carotenoids (e.g., alpha-carotene, astaxanthin, beta-carotene, canthaxanthin, lutein, lycopene, zeaxanthin, etc.), flavonoids (e.g., apigenin, luteolin, tangeritin, isohamnetin, kaempferol, myricetin, proanthocyanids, quercetin, eriodictyol, hesperetin, naringenin, catechin, gallocatechin, epicatechin, epogallocatechin, thealflavin, thearubigins, daidzein, genistein, glycitein, reservatrol, pterostilbene, cyaniding, delphinidin, malvidin, pelargonidin, peonidin, petunidin, etc.) phenolic acids and their esters (e.g., chicoric acid, chlorogenic acid, cinnamic acid, ellagic acid, ellagitannins, gallic acid, gallotannins, rosmarinic acid, salicylic acid, curcumin, silymarin, xanthones, eugenol, etc.) capsaicin, bilirubin, citric acid, oxalic acid, phytic acid, n-acetylcysteine, lipoic acid, uric acid, etc. Each of the aforementioned antioxidants are commercially available from a wide range of sources. In particular embodiments of the present invention, the selection of antioxidants to include in the additives and supplements can be based on the effects that a given antioxidant has on liver function or protection and the solubility of the antioxidant in alcoholic and aqueous environments.

Lipotropic compounds, or lipotropics, include compounds that help catalyze the breakdown of fat during metabolism in the body. Non-limiting examples of lipotropics that can be used with the additives and supplements of the present invention include choline and inositol (which includes their salt forms too). Each of these ingredients are commercially available from a wide range of sources (e.g., Sigma-Aldrich Co. LLC, St. Louis, Mo.). Additional non-limiting examples of lipotropic compounds that can be used in the context of the present invention include methionine, folic acid, betaine or betaine hydrochloride, each of which are also commercially available from a wide range of sources. In particular embodiments of the present invention, the selection of lipotropics to include in the additives and supplements can be based on the effects that a given lipotropic compound has on liver function or protection and the solubility of the lipotropic in alcoholic and aqueous environments.

Water-soluble vitamins include vitamins that are soluble in water. Non-limiting examples of water-soluble vitamins that can be used with the additives and supplements of the present invention include any one of or any combination of or all of the B vitamins (i.e., vitamin B₁ (i.e., thiamine), vitamin B₂ (i.e., riboflavin), vitamin B₃ (i.e., niacin or niacinamide), vitamin B₅ (i.e., pantothenic acid), vitamin B₆ (i.e., pyridoxine, pyridoxamine, pyridoxal, etc.), vitamin B₇ (i.e., biotin), vitamin B₉ (folic acid, folinic acid, etc.), and vitamin B₁₂ (i.e., cyanocobalamin, hydroxycobalamin, methylcobalamin, etc.), and vitamin C (i.e., ascorbic acid). Each of the B vitamins and vitamin C are commercially available from a wide range of sources (e.g., Sigma-Aldrich Co. LLC, St. Louis, Mo.). In particular embodiments of the present invention, the selection of water-soluble vitamins to include in the additives and supplements can be based on the effects that a given waters-soluble vitamin has on liver function or protection and the solubility of the water-soluble vitamin in alcoholic and aqueous environments.

Plants and extracts thereof that can be used with the additives and supplements of the present invention include those that can have beneficial effects on liver function or protection. In particular instances, Silybum marianum or Camellia sinensis, or extracts thereof, can be included in the additives or supplements. Silybum marianum (also known as milk thistle) is a plant native to Southern Europe and Asia. It is known for producing red to purple flowers, shiny pale green leaves with white veins, and fruit. An actives ingredient in this plant, and extracts, thereof is silymarin (silymarin is a mixture of flavanonol derivatives that includes silibine, silicristine, silidianin, isosolibine, and isosilicristine). The fruit portion of Silybum marianum includes silymarin. Silybum marianum extract can be obtained from the fruit portion of this plant by macerating the fruit pulp and then subjecting the pulp to a hydro alcoholic solution of water and SD alcohol 39-C (alcohol denat.) to obtain the extract. The extract can then filtered and packaged for storage or be added to a composition of the present invention. In addition to this extraction process, Silybum marianum extract can be purchased from Provital S.A (SPAIN) under the trade names PRONALEN SILYMARIN HSC or PRONALEN SILYMARIN SPE. As for Camellia sinensis (also known as green tea), it is a flowering plant that is native to China. This plant includes actives ingredients such as a polyphenol compound known as epigallocatechin gallate. Extracts from each of Silybum marianum and Camellia sinensis can be obtained from the whole plant or parts of said plant. Camellia sinensis extracts are commercially available from a wide range of sources (see, e.g., CTFA, Volume 1, pages 400-07, which is incorporated by reference). Another possible plant that can be used in the context of the present invention includes the Vitis vinifera (grape) plant, which is a vine that is native to the Mediterranean region, central Europe, and southwestern Asia. In particular instances, the seed portion can be used. Extracts of the seed is commercially available from a wide range of sources (see, e.g., CTFA, Volume 3, pages 2891-93, which is incorporated by reference). This extract can include polyphenols such as catechin and epicatechin and flaconoids. Additional plants and extracts thereof that have beneficial or protective properties for liver function and health can also be used in the context of the present invention.

With respect to the plants and extracts thereof, either the whole plant can be used or parts of the plant can be used (e.g., leaf, root, flower, fruit, seed, bark, etc.). With respect to Silybum marianum, and in particular instances, the extract can be obtained from the fruit portion. With respect to Camellia sinensis, the extract can be obtained from the leaf portion. With respect to Vitis vinifera, the extract can be obtained from the seed portion.

Further, plant materials can be processed by obtaining the whole plant (or any part of the plant) and disrupting it by mechanical means which results in a puree. The puree can then processed to be substantially free of impurities or undesired solids. The puree can then be poured into a shallow vessel and quickly exposed to low temperature, i.e., flash frozen, for example at −20° C. or lower, preferably under a vacuum for removal of water content (lyophilization). The resultant extract can then be used in the additives or supplements of the present invention. Alternatively, extracts can be obtained by subjecting the plant material (or part thereof) to aqueous, alcoholic, or aqueous/alcoholic extraction techniques, or combinations thereof. Such extracts can then be used in the additives or supplements of the present invention. Extraction techniques such as those mentioned are well-known to persons having ordinary skill in the art. For instance, such processes include maceration, infusion, percolation, digestion, decoction, hot continuous extraction, aqueous-alcoholic extract, counter current extract, microwave assisted extraction, ultrasound extraction, supercritical fluid extracts, phytonic extract (e.g., with hydro-flouro-carbon solvents), etc.

Fat-soluble vitamins include vitamins that are soluble in hydrophobic environments such as hydrocarbons or oil. Non-limiting examples of fat-soluble vitamins that can be used with the additives and supplements of the present invention include any one of or any combination of or all of the A vitamins (i.e., retinol, retinal, retinoic acid, and provitamin A carotenoids), D vitamins (i.e., vitamin D₂, vitamin D₃), E vitamins (i.e., tocopherols, tocotrienols, preferably α-tocopherol), and K vitamins (i.e., vitamin K₁ and vitamin K₂). Each of these vitamins are commercially available from a wide range of sources (e.g., Sigma-Aldrich Co. LLC, St. Louis, Mo.). In particular embodiments of the present invention, α-tocopherol and acetate salts thereof are preferred. The selection of fat-soluble vitamins to include in the additives and supplements of the present invention can be based on the effects that a given fat-soluble vitamin has on liver function or protection and the solubility of the fat-soluble vitamin in alcoholic and aqueous environments.

S-Adenosylmethionine (SAMe) is a compound that can be found in tissue and fluid in the body. It can play a role in the immune system, maintains cell membranes, and help produce and break down brain chemicals, such as serotonin, melatonin, and dopamine. It can work with vitamin B₁₂ and folate (vitamin B₉). Vitamin B₁₂ or folate deficiencies can reduce levels of SAMe in a person's body.

### B. Preparation of the Additives and Supplements

The additives and supplements of the present invention can be prepared by methods known in the art. By way of example, each of the ingredients to include in the additive can be placed into an aqueous environment under constant mixing via mechanical mixers (e.g., counter-rotating side-scrapping mixers, homogenizers and dispersers, including in-line or in-tank rotor-stator homogenizers, and mills, including 3-roll mills, rotor-stator mills, etc.). An “all-in-one” vacuum mixing system having a rotating side-scrapping mixer plus an in-tank homogenizer may also be used. Such mixers include, but are not limited to OLSA mixers, FRYMA-KORUMA mixers, and LEE TRI-MIX TURBO-SHEAR kettles. The mixture can be performed under heat to obtain complete solubilization of each ingredient in the aqueous environment. In particular instances, the heat can be less than 100° C. so as to avoid boiling of the water. The solution can then be processed by using spray-drying techniques, freeze-drying techniques, or lyophilization techniques. Spray-drying techniques are well known to those skilled in the art. Spray-drying includes the steps of atomization of a solution containing one or more solutes (e.g., liver active ingredients, excipients, etc.) via a nozzle spinning disk, or other device, followed by evaporation of the solvent from the droplets. The nature of the powder that results is the function of several variables processing parameters, including the initial solute concentration, size distribution of droplets produced and the rate of solute removal. The particles produced may comprise aggregates of primary particles which consist of crystals and/or amorphous solids depending on the rate and conditions of solvent removal. A spray-drying process for preparing ultra-fine powders of is described in, for example, U.S. Pat. No. 6,051,256. Freeze-drying procedures are well-known in the art, and are described, for example, in U.S. Pat. No. 4,608,764 and U.S. Pat. No. 4,848,094. Spray-freeze-drying processes are described, e.g., in U.S. Pat. No. 5,208,998. Other spray-drying techniques are described, for example, in U.S. Pat. Nos. 6,253,463; 6,001,336; 5,260,306; and PCT International Publication Nos. WO 91/16882 and WO 96/09814.

Lyophilization techniques are well-known to those skilled in the art. Lyophilization is a dehydration technique that takes place while a product is in a frozen state (ice sublimation under a vacuum) and under a vacuum (drying by gentle heating). These conditions stabilize the product, and minimize oxidation and other degradative processes. The conditions of freeze drying permit running the process at low temperatures, therefore thermally labile products can be preserved. Steps in freeze drying include pre-treatment, freezing, primary drying and secondary drying. Pre-treatment includes any method of treating the product prior to freezing. This may include concentrating the product, formulation revision (i.e., addition of components to increase stability and/or improve processing), decreasing a high vapor pressure solvent or increasing the surface area. Methods of pre-treatment include: freeze concentration, solution phase concentration, and formulating specifically to preserve product appearance or to provide lyoprotection for reactive products, and are described, e.g., in U.S. Pat. No. 6,199,297. “Standard” lyophilization conditions, are described, e.g., in U.S. Pat. No. 5,031,336, and in “Freeze Drying of Pharmaceuticals” (DeLuca, Patrick P., J. Vac. Sci. Technol., Vol. 14, No. 1, January/February 1977); and “The Lyophilization of Pharmaceuticals: A Literature Review” (Williams, N. A., and G. P. Polli, Journal of Parenteral Science and Technology, Vol. 38, No. 2, March/April 1984). The resulting powder can then be further processed by skills known in the art to produce an additive or supplement in tablet form, liquid form, powdered form, pastes, granules, syrups, lozenges, capsules (e.g., gel caps), or the like.

The amount of each ingredient within the additives and supplements of the present invention (e.g., essential amino acids, non-essential amino acids, antioxidants, lipotropic compounds, water-soluble vitamins, or plants or extracts thereof) can vary as desired to achieve a given result. The amount, by weight of each ingredient, can range such that the additive or supplements include “x” milligrams of each ingredient. For example, each ingredient in the additive can individually be present in the following amounts (in milligrams), 0.0001, 0.001, 0.01, 0.1, 0.5, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 21, 22, 23, 24, 25, 26, 27, 28, 29, 30, 35, 40, 45, 50, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more milligrams.

### C. Use of the Additives and Supplements

The additives and supplements of the present invention can be used in (e.g., additives) or in conjunction with (e.g., supplements) edible compositions such as solid foods, semi-solid foods, or liquid drinks or beverages. In particular instances, the additives and supplements can be used in or alone with such edible compositions where the underlying edible composition (i.e., without the additive) can have or is known to have deleterious effects on a person's liver function or health. One such example is alcoholic beverages, where the alcohol within the beverage can lead to hepatopathy. By incorporating the additive in to an alcoholic beverage or using the supplement prior to, during, or after consumption of the alcoholic beverage, alcoholic induced hepatopathy can be reduced or avoided all together. In this sense, the additives and supplements of the present invention can be thought of as a liversaver in that the health and function of the liver can be protected via the inclusion of the additives in a given alcoholic beverage or via the ingestion of the supplement prior to, simultaneously with, or after ingestion of the beverage. The person ingesting the alcoholic beverage can have a package or tablet that has the additive or supplement. With respect to additives, the additive can be placed in the beverage followed by mixing the beverage (e.g., with a straw) and drinking or consuming the beverage. Alternatively, a supplement can be taken prior to, simultaneously with, or after consuming the beverage (or any combination thereof). This can help protect the liver from the alcohol within the beverage. Alternatively, the manufacturer of the alcoholic beverage or liquor used to make the alcoholic beverage can incorporate the additive into its manufacturing processes to produce an alcoholic beverage that requires to pre-mixing by the end user/consumer of the alcoholic beverage.

In addition to edible compositions, medicinal compositions that are ingested can also benefit from the additives and supplements of the present invention. By way of example, many drugs within medicinal formulations are known to have adverse effects on the liver to one degree or another. Examples of such drugs include those that may cause acute dose-dependent liver (e.g., acetaminophen), those that may cause acute dose-independent liver damage (e.g., acebutolol, indomethacin, phenylbutazone, allopurinol, isoniazid, phenytoin, atenolol, ketoconazole, piroxicam, carbamazepine, labetalol, probenecid, cimetidine, maprotiline, pyrazinamide, dantrolene, metoprolol, quinidine, diclofenac, mianserin, quinine, diltiazem, naproxen, ranitidine, enflurane, para-aminosalicylic acid, sulfonamides, ethambutol, penicillins, sulindac, ethionamide, phenelzine, tricyclic antidepressants, halothane, phenindione, valproic acid, ibuprofen, phenobarbital, verapamil), those that may cause acute fatty infiltration of the liver (e.g., adrenocortical steroids, phenothiazines, sulfonamides, antithyroid drugs, phenytoin, tetracyclines, isoniazid, valproic acid, methotrexate), cholestatic jaundice (e.g., actinomycin D, chlorpropamide, erythromycin, amoxicillin/clavulanate, cloxacillin flecainide, azathioprine, cyclophosphamide, flurazepam, captopril, cyclosporine, flutamide, carbamazepine, danazol, glyburide, carbimazole, diazepam, cephalosporins, disopyramide, griseofulvin, chlordiazepoxide, enalapril, enalapril, haloperidol, ketoconazole, norethandrolone, sulfonamides, mercaptopurine, oral contraceptives, tamoxifen, methyltestosterone, oxacillin, thiabendazole, nifedipine, penicillamine, tolbutamide, nitrofurantoin, phenothiazines, tricyclic antidepressants, nonsteroidal, phenytoin troleandomycin, anti-inflammatory drugs, propoxyphene, verapamil), those that may cause liver granulomas (chronic inflammatory nodules), allopurinol, phenytoin, aspirin, hydralazine, procainamide, carbamazepine, isoniazid, quinidine, chlorpromazine, isoniazid, quinidine, chlorpromazine, nitrofurantoin, sulfonamides, diltiazem, penicillin, tolbutamide, disopyramide, phenylbutazone), those that may cause chronic liver disease or those that may cause active hepatitis (e.g., acetaminophen, dantrolene, methyldopa, isoniazid, nitrofurantoin), those that may cause cirrhosis or fibrosis (e.g., methotrexate, terbinafine HCI (Lamisil, Sporanox), nicotinic acid), those that may cause chronic cholestasis (e.g., chlorpromazine/valproic acid (combination), imipramine, thiabendazole, phenothiazines, tolbutamide, chlorpropamide/erythro-mycin (combination), phenytoin), those that may cause liver tumors (benign and malignant) (e.g., anabolic steroids, oral contraceptives, thorotrast, danazol, testosterone), those that may cause damage to liver blood vessels (e.g., adriamycin, dacarbazine, thioquanine, anabolic steroids, mercaptopurine, vincristine, azathioprine, methotrexate, vitamin A (excessive doses), carmustine, mitomycin, cyclophosphamide/cyclo-sporine (combination), oral contraceptives).

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

A proposed formulation for an additive of the present invention is provided in Table 1.

TABLE 1

| Ingredient | Amount | |
|---|---|---|
| Histidine | 300 | (mg) |
| Isoleucine | 500 | (mg) |
| Leucine | 500 | (mg) |
| Methionine | 50 | (mg) |
| Tryptophan | 10 | (mg) |
| Valine | 200 | (mg) |
| Alanine | 300 | (mg) |
| Aspartic acid | 300 | (mg) |
| Cysteine | 30 | (mg) |
| Glutamine | 500 | (mg) |
| Glycine | 100 | (mg) |
| Proline | 200 | (mg) |
| Serine | 300 | (mg) |
| Tyrosine | 200 | (mg) |
| Alpha Lipoic Acid | 30 | (mg) |
| Choline | 10 | (mg) |
| Inositol | 20 | (mg) |
| Vitamin B₁₂ | 500 | (mcg) |
| Silibum marianum extract | 300 | (mg) |
| Camellia sinensis extract | 150 | (mg) |
| Methylsulfonylmethane | 500 | (mg) |
| Vitamin C | 60 | (mg) |
| Folic acid | 400 | (mcg) |
| Zinc acetate | 15 | (mg) |

The formulation in Table 1 can be prepared by adding each ingredient into a vessel comprising water during constant mixing so as to solubilize each ingredient and to create a clear homogenous solution. The water can be heated to a temperature of approximately 80° C. to less than 100° C. to aid in solubilizing each ingredient. After a clear homogenous solution is obtained, the solution can then be cooled down to room temperature (20° C. to 25° C.). The solution can then be dried via a spray-drying technique to obtain a powder that includes each of the ingredients in Table 1. The powder is an additive that can be used in the context of the present invention to prevent or reduce hepatopathy, and especially alcoholic hepatopathy, by mixing the additive with a food, beverage, or medicinal composition.

### Example 2

A proposed formulation for a supplement of the present invention is provided in Table 2.

TABLE 2

| Ingredient | Amount | |
|---|---|---|
| Alpha Lipoic Acid | 10 | (mg) |
| Beta Carotene | 5 | (mg) |
| Choline | 10 | (mg) |
| Curcumin | 20 | (mg) |
| Cysteine | 30 | (mg) |
| Glutamine | 100 | (mg) |
| Glycine Taurine mixture | 100 | (mg) |
| Folic Acid | 250 | (mcg) |
| Camellia sinensis extract | 200 | (mg) |
| Inositol | 10 | (mg) |
| Methylsulfonylmethane | 100 | (mg) |
| Silibum marianum extract | 250 | (mg) |
| s-adenosylmethionine | 100 | (mg) |
| Vitamin C | 60 | (mg) |
| Vitamin E | 30 | (IU) |
| Zinc gluconate | 5 | (mg) |

The formulation in Table 2 can be prepared by adding each ingredient into a vessel comprising water during constant mixing so as to solubilize each ingredient and to create a clear homogenous solution. The vitamin E can partially solubilize or be dispersed throughout the solution. The water can be heated to a temperature of approximately 80° C. to less than 100° C. to aid in solubilizing each ingredient. After a clear homogenous solution is obtained, the solution can then be cooled down to room temperature (20° C. to 25° C.). The solution can then be dried via a spray-drying technique to obtain a powder that includes each of the ingredients in Table 2. The powder is a supplement that can be used in the context of the present invention to prevent or reduce hepatopathy, and especially alcoholic hepatopathy, by orally ingesting the supplement prior to alcohol consumption, during alcohol consumption, or after alcohol consumption. In particular aspects, the Table 2 formulation is in the form of a gel cap and taken daily, irrespective if the user has an alcoholic beverage for a given day. On days that the user has or plans to have an alcoholic beverage, the user can take one gel cap in the morning, then 1 gel cap 2-4 hours before alcohol consumption, and then 1 gel cap 2-4 hours after alcohol consumption. In particular aspects, the user should take no more than 4 gel caps or doses per day.

The gel cap can be prepared by first adding a liquid (e.g., water) to the produced powder to form liquid composition. An empty gelatin capsule can be used, which are commercially available from a wide range of sources (see, e.g., Capsugel® Belgium NV, Netherlands; Capsule Depot® Inc., Ontario Canada). The gel cap can be opened (it can have a slightly larger portion which slides over a smaller portion), and the liquid composition can be added to the smaller portion of the capsule (e.g., by a device such as an eyedropper). The larger half of the gel cap can then be slid over the small portion to close the liquid composition within the gel cap. The produced gel cap can then be stored at room temperature or under refrigerator conditions or any type of conditions that help to preserve the liquid composition.

Alternatively, the gel cap can be prepared by adding the produced powder to the empty gelatin capsule by placing the powder in the smaller portion of the capsule and sliding the larger portion over the small portion to close the powder within the gel cap. The produced gel cap can then be stored at room temperature or under refrigerator conditions or any type of conditions that help to preserve the powder within the gel cap.

1. An ingestible supplement capable of promoting liver function, promoting liver health, or reducing alcoholic hepatopathy in a subject, the supplement comprising:
(a) at least one non-essential amino acid;
(b) at least one antioxidant;
(c) at least one lipotropic compound;
(d) at least one water-soluble vitamin; or
(e) at least one plant or extract thereof.

2. The ingestible supplement of claim 1, comprising at least one ingredient from each of (a) to (e).

3. The ingestible supplement of claim 2, comprising at least two non-essential amino acids selected from alanine, arginine, aspartic acid, cysteine, glutamic acid, glutamine, glycine, proline, serine, tyrosine, asparagine, or selenocysteine.

4. The ingestible supplement of claim 3, comprising cysteine, glutamine, and glycine.

5. The ingestible supplement of claim 4, comprising 25 mg to 35 mg of cysteine, 75 mg to 125 mg of glutamine, and 1 mg to 125 mg of glycine or 75 mg to 125 mg of a mixture comprising glycine and taurine.

6. The ingestible supplement of claim 5, wherein the supplement comprises a combination of non-essential amino acids that consists of cysteine, glutamine, and glycine.

7. The ingestible supplement of claim 1, wherein the at least one antioxidant comprises alpha lipoic acid, beta carotene, and curcumin.

8. The ingestible supplement of claim 7, comprising 5 mg to 15 mg of alpha lipoic acid, 3 mg to 7 mg of beta carotene, and 15 mg to 25 mg of curcumin.

9. The ingestible supplement of claim 1, wherein the at least one lipotropic compound is choline or inositol or a combination thereof.

10. The ingestible supplement of claim 9, comprising 5 mg to 15 mg of choline and 5 mg to 15 mg of inositol.

11. The ingestible supplement of claim 1, wherein the at least one water-soluble vitamin is vitamin C or vitamin B.

12. The ingestible supplement of claim 11, wherein the vitamin B is vitamin B₁₂ or vitamin B₉ or a combination thereof.

13. The ingestible supplement of claim 12, wherein the vitamin B₁₂ is cyanocobalamin and the vitamin B₉ is folic acid.

14.-20. (canceled)

21. The ingestible supplement of claim 1, wherein the supplement is in a tablet or pill form.

22. The ingestible supplement of claim 21, wherein the tablet or film is coated with gelatin.

23. The ingestible supplement of claim 1, wherein the supplement is in a powdered form.

24. The ingestible supplement of claim 1, wherein the supplement is in a liquid form.

25.-33. (canceled)

33. A method of ingesting the ingestible supplement of any one of claims 1 to 32 by a person, the method comprising ingesting the supplement prior to consumption of an alcoholic beverage, during consumption of an alcoholic beverage, or after consumption of an alcoholic beverage.

34.-40. (canceled)

41. An additive capable of promoting liver function, promoting liver health, or reducing alcoholic hepatopathy in a subject, the additive comprising:
(a) at least one essential amino acid;
(b) at least one non-essential amino acid;
(c) at least one antioxidant;
(d) at least one lipotropic compound;
(e) at least one water-soluble vitamin; or
(f) at least one plant or extract thereof,
wherein the additive is capable of being solubilized in an alcoholic beverage.

42. The additive of claim 41, comprising at least one ingredient from each of (a) to (f).

43.-86. (canceled)
